(19) United States
(12) Patent Application Publication
(10) Pub. No.: US 2015/0174364 A1
(43) Pub. Date: Jun. 25, 2015

(54) SOFT TIP CATHETER

(71) Applicant: Smiths Medical ASD, Inc., Rockland, MA (US)

(72) Inventors: J. Michael Kennelly, Keene, NH (US); Geoff Clark, Lempster, NH (US)

(73) Assignee: SMITHS MEDICAL ASD, INC., Rockland, MA (US)

(21) Appl. No.: 14/568,184

(22) Filed: Dec. 12, 2014

Related U.S. Application Data
(60) Provisional application No. 61/918,022, filed on Dec. 19, 2013.

(51) Int. Cl. A61M 25/00

(52) U.S. Cl. CPC A61M 25/008; A61M 25/0053; A61M 2025/0081

(57) ABSTRACT

A catheter has a main portion made from an elongated tube and a soft tip that has softer flexural properties than the tube. The tip is affixed to the distal end of the tube. A reinforcement member is intermittently embedded in the body of the tube to provide kink and shear stress resistance as well as enhanced columnar strength and torque-ability to the catheter. With the flexible soft tip, the catheter is diverted when it meets an obstacle inside the patient to prevent the catheter from harming the patient. Another exemplar soft tip catheter is a one-piece elongated catheter with its distal portion selectively notched. A reinforcement member may be embedded along the length of the one-piece catheter or only along the main body portion. The notch at the distal portion may be a spiral cut or a number of ring cuts, with possibly different pitch, width and cut depth.

### FIELD OF THE INVENTION

The present invention relates to catheters such as regional anesthesia catheters, including epidural catheters that provide local anesthesia to a patient and more particularly an epidural catheter that has a soft tip to prevent injury to the patient when the catheter is inserted into the epidural space of the patient.

### BACKGROUND OF THE INVENTION

Regional anesthetic catheters are used to convey anesthetic agent and/or medicament to the epidural space of the patient. The catheter conventionally comprises a small bore, flexible plastic tube having a tip introduced to the epidural space through a hollow metal needle that is subsequently removed.

The catheter must be sufficiently rigid to enable it to be manipulated and introduced without kinking, but also must be sufficiently flexible to enable it to be diverted, on insertion to the epidural space, without puncturing the dura, or other obstacles therein. There are disclosed in the prior art ways of softening the tip of the catheter. Once such example is disclosed in EP 0348136, assigned to a related entity of the assignee of the instant application. As disclosed in the EP '136, to achieve a soft tip, a plurality of parallel slits are provided about the patient end of the catheter so that the flexibility of the patient end of the catheter increases, and is more easily deflected if it should meet an obstruction on insertion. The slits further provide an outlet whereby medicament may be infused to the patient. The use of slits to increase the flexibility of the catheter at the patient end is also disclosed in U.S. Pat. No. 4,801,297. U.S. Pat. No. 8,206,373 discloses the use of an elongated tubular braid along the shaft of the catheter and the attachment to the braid a distal tip made of a second polymeric material.

There is a need for an improved catheter in general and a regional anesthesia catheter in particular with a soft tip whereby the flexibility of the tip of the catheter does not cause the patient end of the catheter to balloon out when it meets an obstacle as is the case with the above-discussed prior art catheters.

### SUMMARY OF THE PRESENT INVENTION

The present invention is a low cost catheter having a soft tip with a flexibility that prevents the risk of puncturing a patient's vessel, or surrounding tissues of a cavity when the catheter is inserted thereinto. The body of the catheter is designed to be strong and durable with a reinforced wall that provides kink and shear resistance and columnar strength. The material of the catheter is bio-compatible, so that the catheter is suitable for extended human use. The catheter material is also compatible with all regional anesthesia medications, so as not to affect the potency of the drug traversing through the catheter. Moreover, the catheter is adapted to be viewable under ultrasound and is compatible with magnetic resonance imaging (MRI), so that the insertion of the catheter into the patient may be guided by viewing the catheter with ultrasound. Further, the catheter is adapted to be used for a patient who is undergoing a MRI procedure.

The catheter of the instant invention is an elongated tube that has a soft tip that has flexural properties that are softer than the main body of the catheter. The soft tip may be a tubing separately formed from the main body of the catheter, and is joined to the distal end of the main body by any of a number of different methods, to be discussed infra. A reinforcement means is embedded into the wall of the catheter, at least with respect to its main body, in an intermittent fashion along the length of the catheter to provide kink and shear resistance and also high columnar strength to the catheter. As a result, the catheter has a rigidity that complements the flexibility of its soft tip. The reinforcement means may be a member that may be made of a nonmetallic material that is compatible with regional anesthesia medications and viewable under ultrasound. Such nonmetallic material may include any of a number of polymeric materials including nylon. The reinforcement member may take the form of a continuous strip or coil that forms within and along the length of at least the main body of the catheter in a spiral fashion. For certain embodiment, the reinforcement member may include continuous strands of polymeric materials and an elongated tubular braid such as that disclosed in the afore-noted U.S. Pat. No. 8,206,373, the disclosure of which is incorporated by reference herein.

The soft tip may be affixed or fixedly attached to the main body of the catheter by over molding the proximal end of the soft tip about the distal end of the catheter body, the welding of the proximal end of the soft tip to the distal end of the main body, or the bonding of the soft tip to the catheter body by any number of conventional bonding means and methods. An innovative way of affixing the soft tip to the catheter is by using a 3-D (three dimensional) printer to add, or print, the material that makes the soft tip to the distal end of the main body of the catheter. also, the soft tip may be an extension of the catheter body.

The main body of the catheter of the instant invention may have a hardness of a given durometer (XX-XX Durometer Shore D), and the soft tip may have a lower durometer than that the main body portion, i.e., the soft tip has flexural properties that are softer than the main body, so that the soft tip is adapted to flex relative to the main body, or orthogonally relative to the longitudinal axis of the catheter. The reinforcement member added to the main body enhances the pushability of the main body and prevents the catheter from kinking and shearing.

The instant invention is therefore directed to an improved catheter, comprising: an elongated tube having a circumferential wall defining at least one lumen terminating at a distal end having an opening to the lumen; at least one reinforcement means intermittently embedded in the wall of the tube to provide a high columnar strength for the tube; a tip portion having softer flexural properties than the tube affixed to the distal end of the tube; wherein the tip portion flexes relative to the tube when it comes into contact with a surface of a vessel or an obstacle in a cavity of a patient whereinto the catheter is inserted.

The instant invention is also directed to an improved catheter, comprising: an elongated tube having a circumferential wall defining at least one lumen terminating at a distal end having an opening to the lumen, a reinforcement means intermittently embedded in the wall to provide additional rigidity to the tube to enhance the pushability, or maneuverability, of the tube, a tip portion having softer flexural properties than the tube and a proximal end affixed to the distal end of the tube, the tip portion flexes relative to the tube to divert the catheter when it comes into contact with an obstacle along its path.

The improved catheter of the instant invention may be manufactured in accordance to the following method steps of: a) forming an elongated tube having a circumferential wall defining at least one lumen terminating at a distal end having an opening to the lumen; b) embedding a reinforcement means within the wall of the tube to provide rigidity and columnar strength for the tube; c) forming a tip portion having softer flexural properties than the tube; and d) affixing the tip portion to the distal end of the tube; wherein the tip portion flexes relative to the tube to divert the catheter when it comes into contact with a surface of a vessel or an obstacle in a cavity of a patient whereinto the catheter is inserted.

The present invention is moreover directed to an improved catheter comprising: an elongated tube having a main body and a distal body along a longitudinal axis, the tube having a circumferential wall formed from an elastomeric material defining at least one lumen terminating at a distal end having an opening to the lumen, the wall having an outer circumferential surface and an inner circumferential surface, at least one reinforcement member embedded in the wall along at least the length of the main body, the wall at the distal body having portions therealong selectively weakened to provide the distal body with flexural properties that are softer than the main body.

The present invention is also directed to an improved catheter comprising: an elongated tube having a main body and a distal body along a longitudinal axis, the tube having a circumferential wall formed from an elastomeric material defining at least one lumen terminating at a distal end having an opening to the lumen, the wall having an outer circumferential surface and an inner circumferential surface, at least one reinforcement member embedded in the wall of the tube, the wall at the distal body having portions of the wall therealong orthogonal to the longitudinal axis of the tube selectively weakened without cutting into the inner circumferential surface to thereby effect the distal body to have flexural properties that are softer than the main body.

The instant invention is furthermore directed to making an improved catheter comprising the steps of: a) forming an elongated tube having a main body, a distal body and a circumferential wall formed of an elastomeric material having an outer circumferential surface and an inner circumferential surface defining at least one lumen terminating at a distal end having an opening to the lumen; b) embedding at least one reinforcement member in the wall about at least the length of the main body; and c) selectively weakening portions of the wall at the distal body to effect the distal body to have flexural properties softer than the main body; wherein the distal body flexes relative to the main body to divert the catheter to a different direction when the catheter comes into contact with an obstacle as it is maneuvered within the patient.

### BRIEF DESCRIPTION OF THE FIGURES

The present invention will become apparent and the invention itself will be best understood with reference to the following description of the instant invention taken in conjunction with the accompanying drawings, wherein:

FIG. 1 is a perspective view of an embodiment of the catheter of the instant invention;

FIG. 2 is a cross sectional view of the patient end portion of the FIG. 1 catheter;

FIG. 3 is a cross sectional view of the patient end portion of the inventive catheter with the soft tip having an atraumatic end with multiple ports;

FIG. 4 is a cross sectional view of the patient end portion of the inventive catheter with the soft tip having an atraumatic end and side ports provided at the distal end of the main body of the catheter;

FIG. 5 shows the patient end portion of the inventive catheter with the soft tip over molded to the distal end of the main body of the catheter;

FIG. 6 shows an alternative exemplar catheter of the instant invention;

FIG. 7 is a cross sectional view of a variant of the patient end portion of the catheter shown in FIG. 6;

FIG. 8 is a cross sectional view of the patient end portion of another variant of the catheter shown in FIG. 6;

FIG. 9 is a perspective view of the patient end portion of a catheter that has a spiral cut, variable pitch configuration;

FIG. 10 is a cross-sectional view of the patient end portion of FIG. 9;

FIG. 11 is a patient end portion of a catheter having side ports that has a spiral cut, variable pitch configuration;

FIG. 12 is a cross-sectional view of the FIG. 11 patient end portion;

FIG. 13 is a variant of the patient end portion of the instant invention that has a ring cut, variable width variation;

FIG. 14 is a cross-sectional view of the FIG. 13 patient end portion;

FIG. 15 is another variant of the patient end portion of the catheter with side ports that has a ring cut, variable width configuration;

FIG. 16 is a cross-sectional view of the FIG. 15 patient end portion;

FIG. 17 is yet another variant of the patient end portion of the instant invention having a ring cut, variable width and depth configuration;

FIG. 18 is a cross-sectional view of the FIG. 17 patient end portion;

FIG. 19 is a further variant of the patient end portion of a side port catheter of the instant invention having a ring cut, variable width and depth configuration; and

FIG. 20 is a cross-sectional view of the FIG. 19 patient end portion.

### DETAILED DESCRIPTION OF THE INVENTION

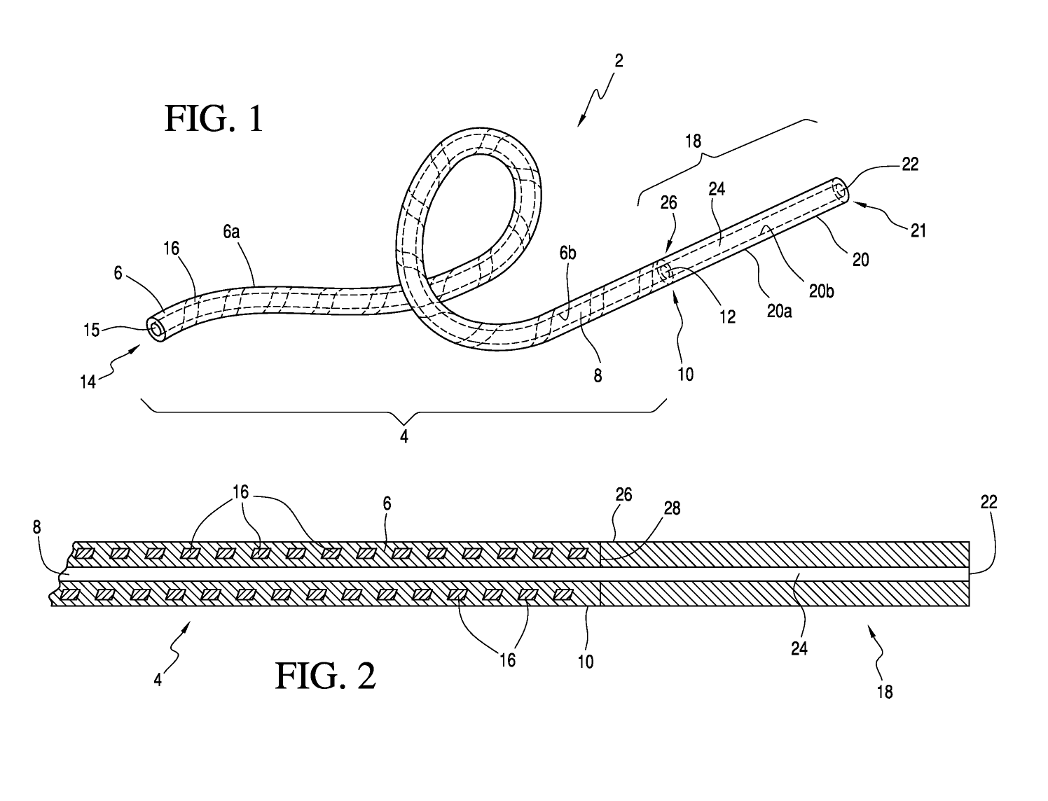

FIG. 1 shows a perspective view of an exemplar catheter 2. As shown, catheter 2 is an elongated tube 4 that has a circumferential wall 6 that defines a lumen 8 that terminates at a distal end 10 with an opening 12, represented by dotted lines. Tube 4 also has a proximal end 14 that has an opening 15 that extends through the passage, or lumen 8, to opening 12 at distal end 10. Wall 6 is defined by an outer circumferential surface 6a and an inner circumferential surface 6b. Lumen 8 is defined by inner circumferential surface 6b. The length of the elongated tube 4 can vary, and therefore is not limited by what is shown in FIG. 1. The catheter may have one of its ends integrally connected to a hub (not shown) to enable the catheter to be connected to a syringe, or other fluid source, and/or for the insertion of a needle and/or a guide wire through the catheter as is well known.

Elongated tube 4 may have a number of different sizes, including but not limited to 20 gauge that has O.D. (outside diameter) of approximately 1.05 mm and an I.D. (inside diameter) of approximately 0.55 mm. The catheter embodiment shown in FIG. 1 may also be a 21 gauge tube that has an O.D. of approximately 0.85 mm and an I.D. of approximately 0.44 mm. Yet another size of the catheter might be a 24 gauge where the O.D. is approximately 0.55 mm and the I.D. is approximately 0.30 mm. The elongated tube 4 may have other gauges in actual usage.

There is embedded in wall 6 of tube 4 at least one reinforcement member, shown in the FIG. 1 catheter as a strip 16 that spirally winds or wraps within wall 6 along the length of tube 4. Thus, per shown in the cross sectional view of FIG. 2, the reinforcement member provides reinforcement of the catheter intermittently along the length of the catheter. It was found that such intermittent support provides adequate rigidity and maneuverability for the catheter. Also, having the reenforcement member(s) embedded within the body of the catheter means that both the inner and outer circumferential surfaces of the catheter are smooth. As a result, by embedding a spiral reinforcement member or evenly spaced reinforcement means or members along the body of the catheter, a catheter with good rigidity and maneuverability, as well as having high columnar strength, is able to be more easily manufactured and at a lower cost, as compared for example to the need to provide a tubular braid for the catheter described in the above-noted U.S. Pat. No. 8,206,373. For illustration purposes, reinforcement member 16 is shown in FIG. 1 to be in touch with the outer circumferential surface 6a and the inner circumferential surface 6b. In actuality, as more accurately shown in the cross sectional view of the catheter in FIG. 2, strip 16 is embedded within wall 6 away from the inner and outer circumferential surfaces. Although shown as a strip, reinforcement member 16 may also be a coil or a braided wire-like member. As will be discussed later, instead of being spirally embedded in wall 4, multiple reinforcement members may be embedded longitudinally along the length of the tube. See for example the to be discussed alternative catheter embodiment of FIG. 8.

Given that the catheter of the instant invention is to be inserted to a vessel or cavity of a patient, and in particular if the catheter is an epidural catheter to be inserted through the dura and into the epidural space of a patient, the catheter has to be compatible with human use and also has to have properties that enhance its maneuverability and prevent it from kinking. The reinforcement member embedded in the body of the catheter provides columnar strength and shear resistance for the catheter as well as prevents the catheter from kinking as it is inserted into and maneuvered within the patient. So that the tube is compatible with medicaments and drugs, for example all regional anaesthesia medications, to ensure that the potency of the drug is not altered by passing through the catheter, the tube of the catheter may be made, by extrusion for example, from a number of different medicament compatible materials including nylon, PET (polyester), PU (polyurethane), PEEK (polyether ether ketone), PEKK (polyether ketone ketone), PEI (polyetherimide) and polyimide.

Additional elastomeric materials disclosed in the afore-noted U.S. Pat. No. 8,206,373 may also be used. They include, but are not limited to, polymers such as polyoxymethylene (POM), polybutylene terephthalate (PBT), polyether block ester, polyether block amide (PEBA), fluorinated ethylene propylene (FEP), polyethylene (PE), polypropylene (PP), polyvinylchloride (PVC), polyurethane, polytetrafluoroethylene (PTFE), polyamide, polyphenylene sulfide (PPS), polyphenylene oxide (PPO), polysulfone, nylon, perfluoro(propyl vinyl ether) (PFA), polyether-ester, metals, polymer/metal composites, etc., or mixtures, blends or combinations thereof. One example of a suitable polyether block ester is available under the trade name ARNITEL®, and one suitable example of a polyether block amide (PEBA) is available under the trade name PEBAX®, from ARKEMA of North America, King of Prussia, Pa. One example of a suitable polyoxymethylene (POM) is Delrin™ commercially available from Dow Chemicals.

The catheter of the embodiment in FIG. 1 is further shown to have a tip portion 18 that may be made from the same material as disclosed above but with flexural properties that are softer than those of the elongated tube 4, i.e., the main body of the catheter. As shown, tip portion 18 is an elongated tubing that has substantially the same cross-sectional dimensions as tube 4. Thus, tip portion 18 has a circumferential wall 20 that is defined by an outer circumferential surface 20a and an inner circumferential surface 20b. An opening 22 at the distal end 21 of tip portion 18 defines a lumen 24. As the distal end 10 of tube 4 is connected to the proximal end 26 of tip portion 18, lumen 8 of tube 4 and lumen 24 of tip portion 18 are joined together to form a through passage along the length of catheter, with opening 15 as the inlet and opening 22 as the outlet of the catheter. Proximal end 26 of tubing 18 and distal end 10 of tube 4 are affixed or fixedly attached to each other.

Proximal end 26 of tubing 18 may be affixed to distal end 10 of tube 4 a number of ways, including over molding whereby the inner circumferential wall 20b at the proximal end 26 of the tubing is fitted about and affixed to the outer circumferential wall 6a at the distal end 10 of the tube. This is shown in the exemplar embodiment of FIG. 5. Another way in which tubing 18 and tube 4 may be affixed together is by using conventional welding techniques including ultrasonic and heat welding to weld the proximal end 26 of tubing 18 to the distal end 10 of tube 4. Tubing 18 may also be connected to tube 4 by bonding, for example ultrasonic cold bonding, or gluing. Yet another way in which tubing 18 may become a portion of the catheter is by adding the material that forms tubing 18 to distal end 10 of tube 4 by 3-D (three dimensional) printing, using a 3-D printer. Tubing 18 and tube 4, once affixed to each other, effect an integral one piece unitary catheter. For purpose of illustration, a demarcation line 28 showing the junction where tube 4 is joined to tubing 18 is provided in the cross sectional views of the catheters shown in FIGS. 2-5.

As disclosed above, elongated tube 4 may be made from a plastics material such as nylon, polyether block amide (PEBA), or a blend of the two in some embodiments, and may have a durometer in the order of about 30-100 Durometer Shore D, preferably between 50-80 Durometer Shore D. The soft tip elongated tubing 18 may also be made of a plastics material such as nylon and other similar material as the elongate tube but may have a range of approximately 0-60 Durometer Shore D, preferably between 0-30 Durometer Shore D (or 0-100 Durometer Shore A). The elongated tube and tubing of the catheter may be constructed using any appropriate technique, for example, by extrusion, a heat bonding process, molding, and the like as is conventionally known. In addition to the material noted above, the elongate tube and tip may also be made from other plastics material such as urethane, PTFE and similar plastics material suitable for medical use.

For the exemplar catheter embodiment of FIG. 1, were it made of nylon, elongated tube 4 may have approximately 50-80 Durometer Shore D (durometer) hardness. Tubing 18 that forms the tip portion of the catheter may also be made of nylon but having a range of approximately 25-55 Durometer Shore D (durometer). The optimal durometer for the exemplar elongated tube 4 may be 65-75 Durometer Shore D, and the optimal durometer for the exemplar tubing 18 may be 35-45 Durometer Shore D. The nylon material may be Pebax, a nylon elastomeric material. After fixedly attaching tube 4 and tubing 18 to each other using any one of the affixing techniques disclosed above, the unitary catheter thus formed has a soft tip tubing 18 having flexural properties that are softer than the main body tube 4 of the catheter. The reinforced wall of tube 4 provides good kink and shear resistance as well as torque-ability to enable the catheter to be readily maneuvered into as well as within the patient. Each of tube 4 and tubing 18 may be extruded from conventional extrusion processes, with the reinforcement member being co-extruded at least with the main body of the catheter, i.e., elongated tube 4 as shown in FIGS. 1 and 2.

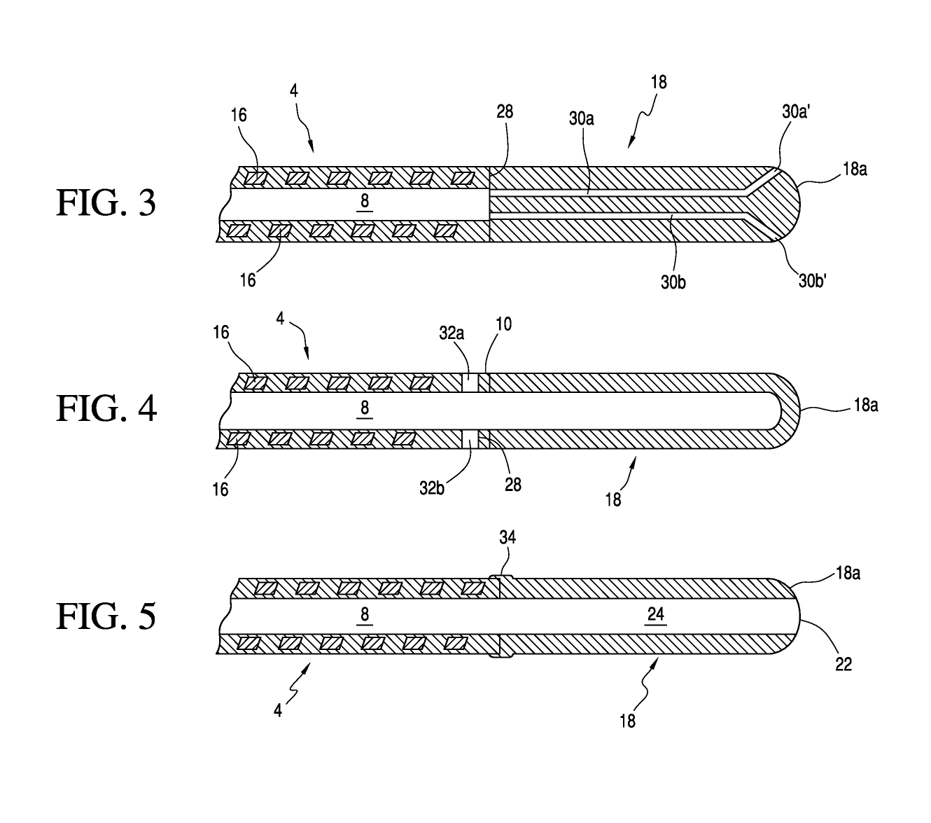

FIG. 3 shows a cross sectional view of the distal portion of an exemplar catheter of the instant invention in which the soft tubing 18 has an atraumatic distal end 18a. A plurality of passages or ports extend through tubing 18 to port openings 30a′ and 30b′, so that tubing 18 of FIG. 3 is a multi-port soft tip of the catheter, as each of ports 30a and 30b is connected to lumen 8 of tube 4.

FIG. 4 shows another exemplar embodiment of the catheter of the instant invention in which soft tubing 18 has an atraumatic closed distal end 18a that prevents the fluid medicament from outputting thereat. For the FIG. 4 embodiment, there is no opening at tubing 18. Instead, a plurality of side ports 32a and 32b are provided at tube 4 proximate to its distal end to enable the outflow of the medicament therefrom. In the FIG. 4 embodiment, tubing 18 may in practice be solid so long as it has sufficient flexibility and has softer flexural properties than tube 4 to prevent potential damage to the patient. Alternatively, side ports 32a and 32b may be provided at tubing 18.

FIG. 5 shows yet another exemplar embodiment of the inventive catheter where the proximal portion of soft tubing 18 over molds to the distal end of tube 4. The over molded portion, designated 34, is exaggeratingly shown in FIG. 5 when, in practice, it is substantially smooth relative to the rest of the catheter. For the FIG. 5 embodiment, the distal end 18a of the soft tip portion is rounded so as to be atraumatic. Moreover, lumen 24 of tubing 18 is in direct fluid communication with lumen 8 of tube 4 so that a through passage extends along the length of the catheter with the medicament exiting at opening 22 at distal end 18a of the soft tubing 18. Another embodiment may also have an atraumatic closed distal end with a plurality of side ports as shown in FIG. 4.

The exemplar catheters shown in FIGS. 1-5 therefore each have a strong and durable main body with a reinforced wall that provides kink and shear resistance as well as high columnar strength to enable good maneuverability for the catheter. The soft tip portion of each of those catheters prevents the catheter from puncturing the veins, or other obstacles when the catheter is inserted into the patient. Moreover, being made from the polymeric materials disclosed above, the catheters are compatible for human use, do not affect the potency of the medicaments used therewith, and are viewable under ultrasound.

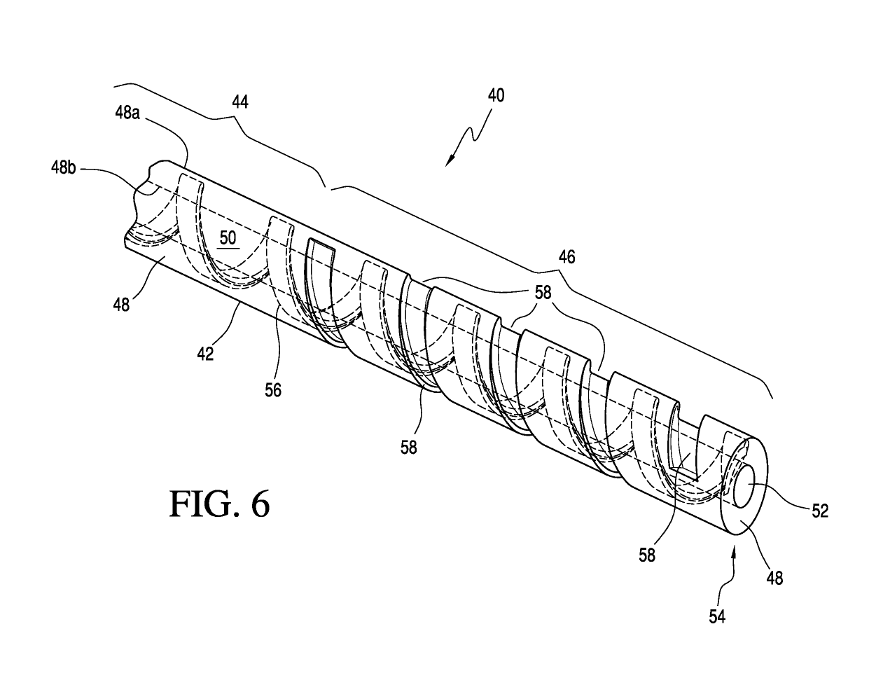

FIG. 6 shows an alternative catheter where there is no need to affix a soft tip to the main body of the catheter. For this alternative embodiment, catheter 40 is made from an elongated tube 42 that has a main body or portion 44 and a distal body or portion 46. Only a small portion of main body 44, which may be of various lengths, is shown in FIG. 6. Catheter 40 is shown to have a circumferential wall 48 that is defined by an outer circumferential surface 48a and an inner circumferential surface 48b. A lumen 50 is defined by the inner circumferential surface 48b and extends throughout the catheter, ending in an opening 52 at a distal end 54. Within wall 48 there is embedded a reinforcement member 56 between outer circumferential surface 48a and inner circumferential surface 48b. As shown, reinforcement member 56 is a strip that spirally winds or wraps about lumen 50 along the length of catheter 40. Other kinds of reinforcement members including a spring-like coil, a wire or the type of braided member as described in the aforenoted U.S. Pat. No. 8,206,373, may also be used.

Instead of winding or wrapping about lumen 50 within wall 48 of the catheter, the reinforcement member may be substituted by a plurality of reinforcers that do not have the spiral formation as shown in FIG. 6. One such alternative embodiment is illustrated in FIG. 8 where the reinforcement members, only two of which (56a and 56b) are shown, are embedded in the body of the catheter longitudinally along the length of the main body of the catheter. However, it should be recognized that in practice, such longitudinal reinforcement members may extend along the length of the catheter, provided that those members do not adversely affect the flexibility of the distal portion of the catheter as will be described in greater detail below.

For the exemplar catheter shown in FIG. 6, distal body 46 eliminates the need for an added soft tip to the catheter, per shown in the FIG. 1 embodiment. This is effected by a continuous notch, groove, slot or cut 58 made along distal body 46 so that material is removed therefrom to thereby cause distal body 46 to be more flexible than the rest of the catheter. The notch, groove, slot or cut 58 in wall 48 begins from outer circumferential surface 48a and extends to a given depth and is such that it does not to come into contact with the reinforcement member 56 embedded in wall 48. Thus, by selectively removing material along the distal body 46, the patient end of the catheter is effected to have flexure properties that are softer than the main body of the catheter. Putting it differently, selective portions of the wall along distal body 46 are weakened due to the removal of material from those portions. Note that instead of removing material at selected portions of the distal body by notching, the weakened portions may result from selective extrusion of less material at the to be distal body of a being extruded elongate tube, or selective reduction of material at the tip portion if the tube is molded. For the sake of simplicity, the notch, groove, slot and cut may summarily be referred to as a notch henceforth.

For the FIG. 6 exemplar catheter, notch 58 is a continuous spiral cut that is in spatial parallel relationship to the reinforcement strip 56 spirally embedded into the wall of the catheter. Thus, with the material removed from distal body 46 due to the spiral groove or notch 58, the notched distal body has flexural properties that are softer than the main body 44.

As is the case with the FIG. 1 embodiment, strip 56 that acts as the reinforcement member may be made from any of the materials disclosed earlier. The depth of notch 58 may be selected to achieve a desired flexibility for distal body 46, which acts as the soft tip relative to main body 44. The manufacturing of catheter 40 may be done by extrusion, with the reinforcement member being extruded along with the elongated tube. The length of the distal body 46 may be variable, and is of a length such that it may act as a soft tip for the catheter. The notch 58 to distal body 46 of catheter 40 may be effected by laser ablation or cutting. Depending on the depth of the notch 58 cut into distal body 46, the flexibility of distal body 46 could be selectively controlled, relative to the hardness or rigidity of main body 44, to adjust the softness of distal body 46. The laser ablation or notching of the distal body of the catheter may be done using a laser machine such as for example an eximer laser machine made by the Coherent Company, Santa Clara, Calif.

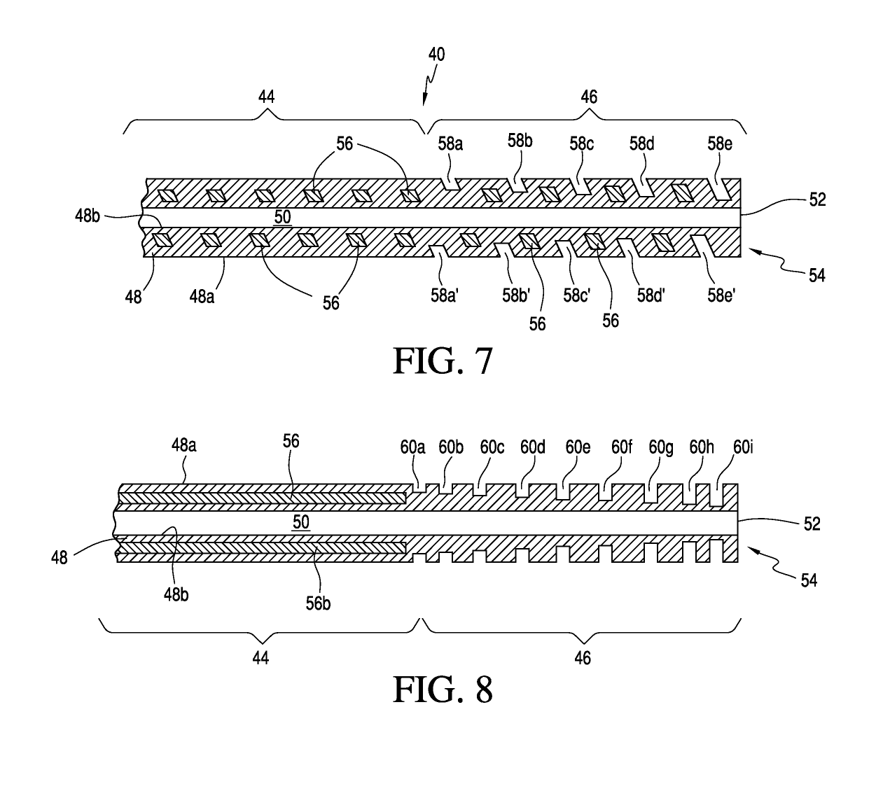

FIG. 7 is a cross sectional view of a catheter that is similar to the FIG. 6 exemplar catheter. For the FIG. 7 catheter, the notch that is made at distal body 46 is shown to have varying depths, so that the flexibility of distal body 46 is selectively varied along the length of the distal body. As shown, notch 58a has a depth that is less than 58b, which in turn has a depth less than 58c, etc. Conversely, notch 58e cut into the body or wall 48 of the catheter proximate to its distal tip has a greater depth than notch 58a proximate to main body 44. The same varying depths are true with respect to the notches 58a′ to 58e′ shown. It should be appreciated however that the various notches shown in the cross sectional catheter of FIG. 7 in fact are formed as one continuous spiral cut, such as that shown in FIG. 6. In any event, given that notch 58e has a greater depth than notch 58d, which in turn has a greater cut depth than 58c, the portion of distal body 46 designated by 58e and 58e′ therefore has a greater flexibility, relative to main body 44, than the portion of distal body 46 that is encircled by notches 58b and 58b′. Thus, as illustrated by the one piece catheter shown in FIG. 7, the flexibility of the soft tip portion of the catheter can be selectively varied along its length.

It should be appreciated that the continuous spiral groove or notch shown in FIGS. 6 and 7 may be replaced by a plurality of circumferential cuts or notches, possibly with different depths, along the length of the distal body 46. In other words, instead of one continuous cut that results in the five exemplar notches shown on each side of the cross section view of the catheter in FIG. 7, there may be a number of cuts or spaces of various widths formed, accordion like, along the length of distal body 46. Also, instead of the depths of the cuts being incremental, the depths of the cuts may vary in no particular order along the length of distal body 46 depending on the kind of flexibility desired for the tip portion of the catheter.

Alternatively, the outer circumferential surface of distal body 46 of the catheter may be shaped to taper from the junction where distal body 46 meets main body 44 to distal end 54, so that distal body 46 may be formed as a tapered cone-shaped extension that has softer flexural properties than main body 44, to thereby allow distal body 46 to flex orthogonally relative to the longitudinal axis of catheter 40, same as the earlier discussed embodiments. In the case where distal body 46 is a cone-shaped tapered extension, there may not be a need to vary the depth of the spiral groove formed at distal body 46, or the need for the reinforcement member to extend to distal body 46 of the catheter, since the distal end of the tapered cone tends to have less material than the base of the cone that starts at the junction where distal body 46 and main body 44 meet.

FIG. 8 shows another variant of the one piece elongated tube catheter of the instant invention. As discussed previously, the reinforcement member for the FIG. 8 exemplar catheter is divided into a plurality of longitudinal members that are embedded along the length of the catheter, at least with respect to its main body 44. Only two of the plurality of longitudinal reinforcement members 56a and 56b are shown to be embedded in wall 48 between outer circumferential surface 48a and inner circumferential surface 48b. These reinforcement members may be made from any of the materials disclosed earlier.

For the FIG. 8 embodiment, instead of one continuous spiral cut, notch or groove, there are now a multiple number of circumferential cuts, notches or grooves, designated 60a to 60i, formed along the length of distal body 46 of the catheter. Same as the FIG. 7 embodiment, the respective depths of the notches may vary. For example, notch 60i is shown to have a deeper cut depth than notch 60a, so that the portion of distal body 46 with notch 60i tends to be the softer, relative to main body 44. As a consequence, the soft tip portion of the FIG. 8 catheter has a flexibility that is greater at its tip and gradually decreases to the junction where distal body 46 meets main body 44.

Even though a gradually increasing depth is shown with respect to the notches from 60a to 60i, the notches may in practice be cut at various depths to selectively control the flexibility of distal body 46 along its length. Further, even through FIG. 8 does not show the reinforcement members extending from main body 44 to distal body 46, in practice, reinforcement strips or coils may actually be provided in distal body 46, possibly in the form of rings for the embodiment shown in FIG. 8, or as a spiral reinforcement member that circumscribes closely about the inner circumferential surface 48b, so as not to be exposed by the notches. The reinforcement member, as discussed above, instead of being a strip, may in fact be made from strands of reinforcement materials, wireless extensions or braided members that are made from materials that are compatible for use with a patient under MRI and would allow the viewing of the catheter under ultrasound as it is being maneuvered and positioned within the patient.

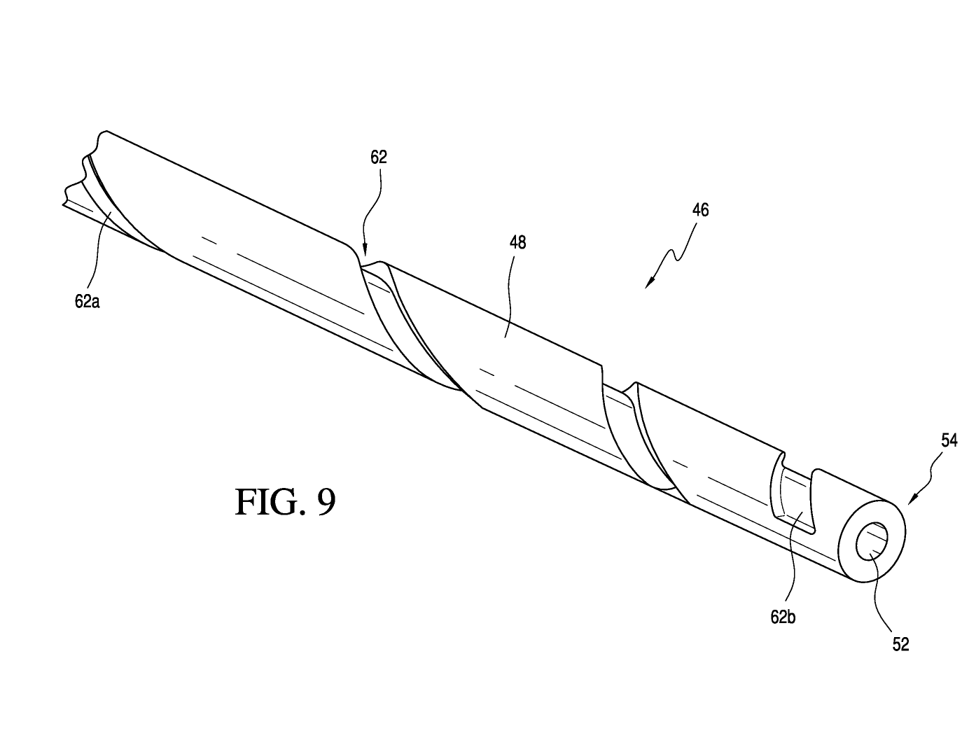

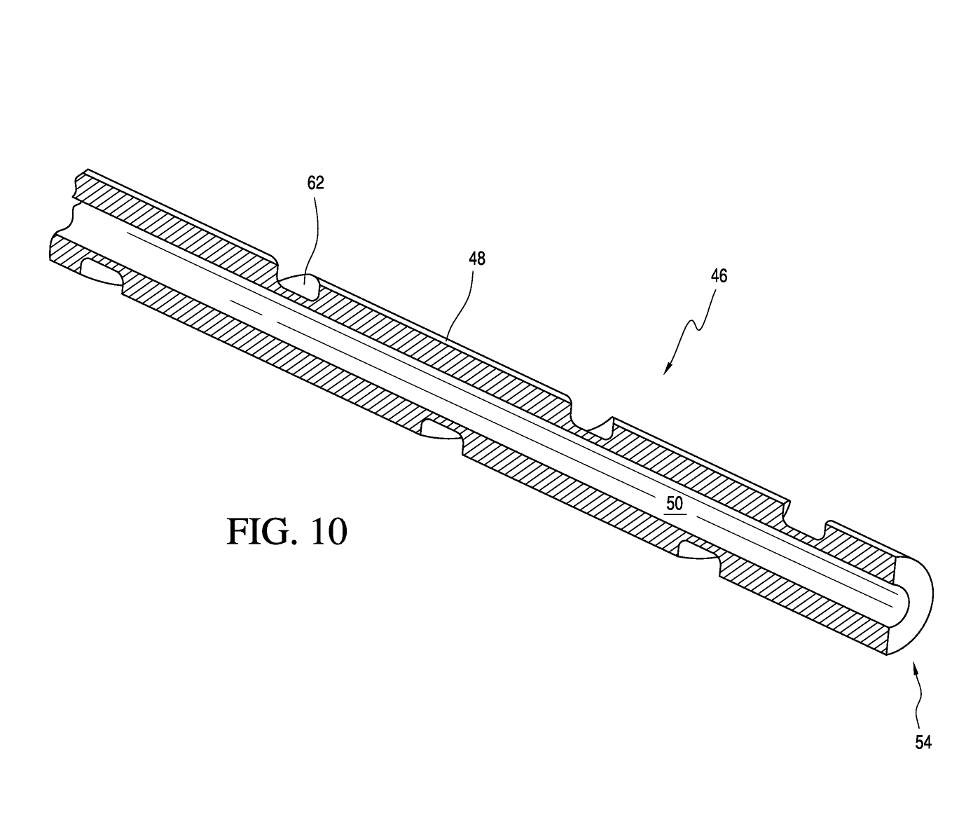

FIG. 9 shows a distal portion of a variant of the catheter of the instant invention in which the notch 62 cut along distal portion 46 has a spiral cut with variable pitch configuration. As shown, the spirally cut notch 62 extends at end 62a proximate to the main body of the catheter at a pitch that gradually increases to end 62b proximate to the catheter end 54, or opening 52. As a consequence, there is more flexibility at the distal portion 46 that is closest to distal end 54, or distal opening 52 for this variant of the catheter. A cross-sectional view of the distal end portion of the catheter is shown in FIG. 10.

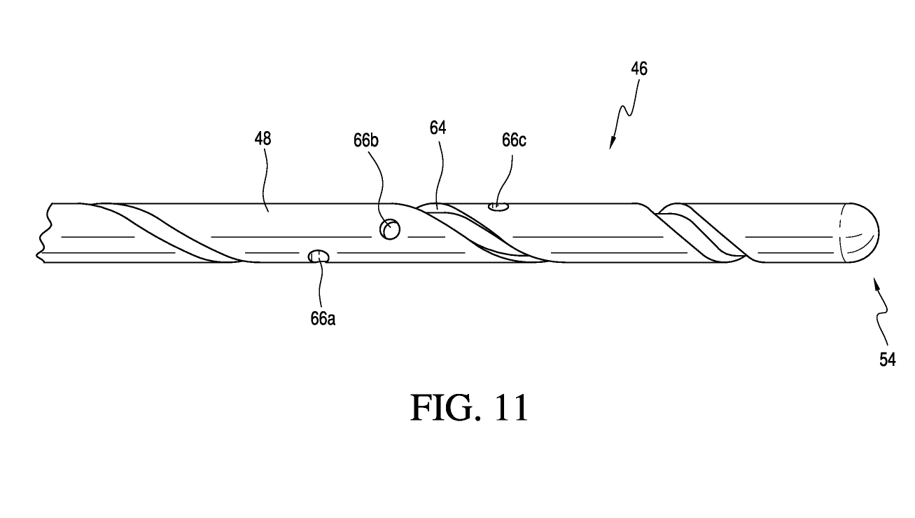

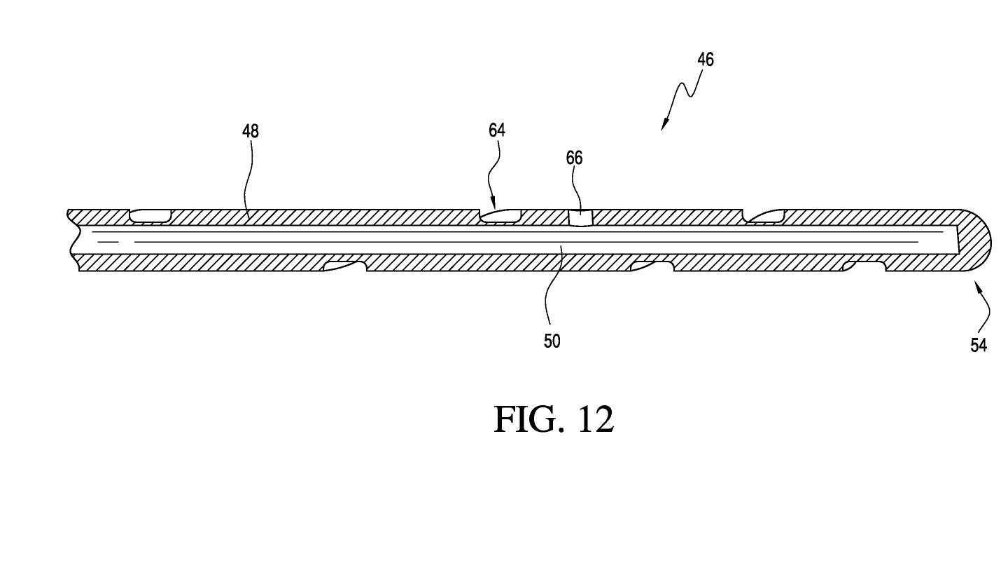

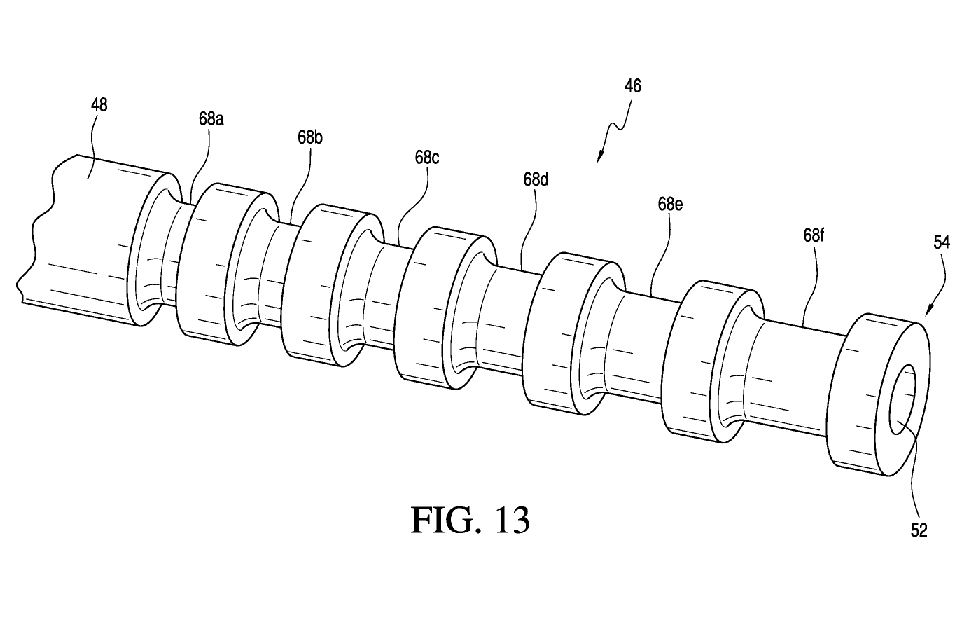

Another variant of the patient end portion of the catheter of the instant invention is shown in FIG. 11. The patient end portion of the catheter of FIG. 11 is similar to that shown in FIG. 9 in that the FIG. 11 patient end portion also has a spirally cut notch having a variable pitch along the distal end portion. But for the FIG. 11 patient end portion, the distal end 54 of the catheter is not open. Instead, the FIG. 11 patient end portion has side ports 66a-66c that allow medicament to output from the catheter. As shown, notch 64 has substantially the same spiral cut and variable pitch as in the FIG. 9 patient end portion. FIG. 12 is a cross-sectional view of the side port patient end portion of FIG. 11.

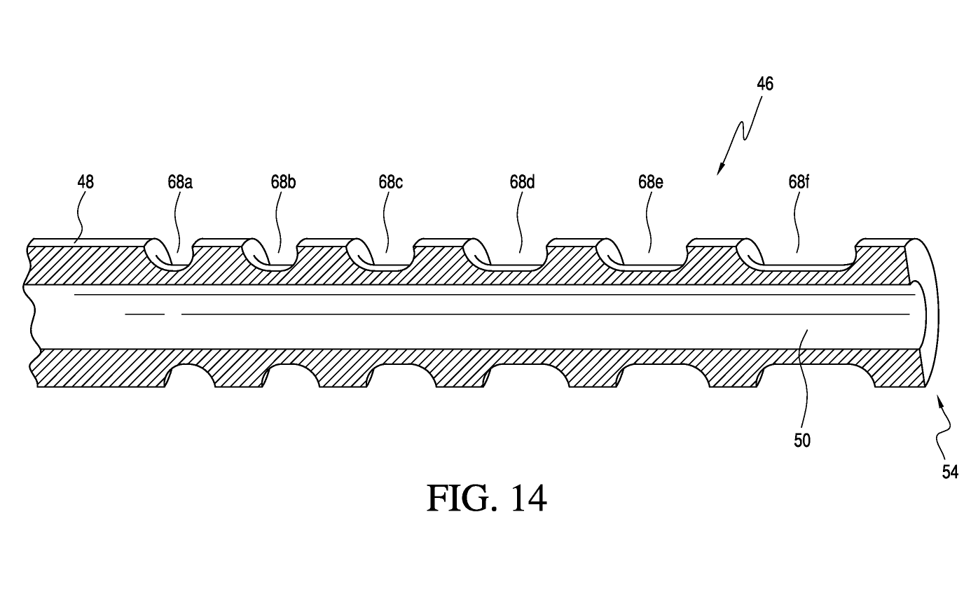

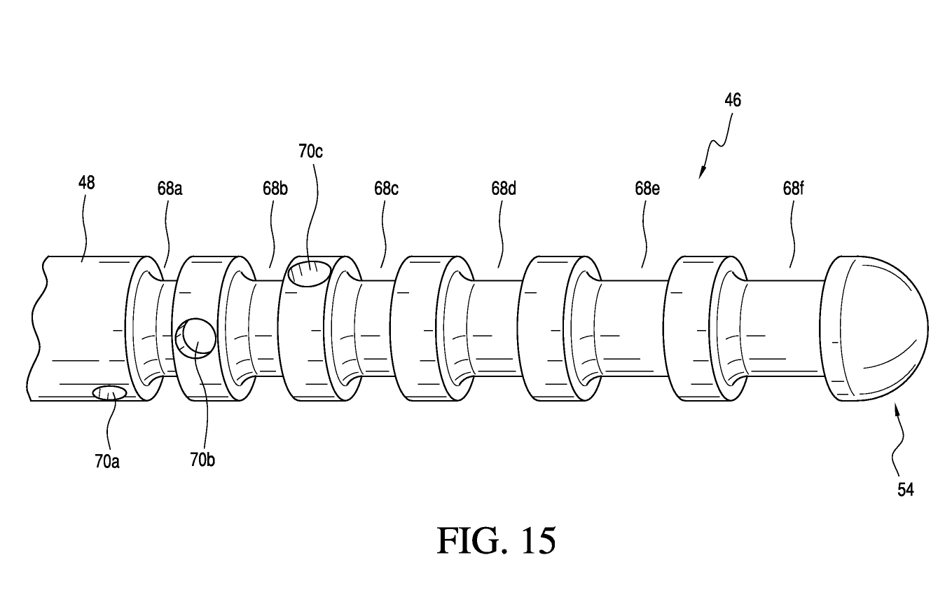

FIG. 13 shows a perspective view of another variant of the patient end portion of the instant invention catheter that has a ring cut, variable width configuration. As shown, there are six cuts or notches 68a-68f at the distal portion 46 of the catheter. Ring cut 68a is a smaller cut in width than ring cut 68b, which in turn has a smaller cut width than ring cut 68c, etc. until the last ring cut 68f, which has the widest width. As a consequence, there is more flexibility at the distal portion 46 that is closest to distal end 54, or distal opening 52 for this variant of the catheter. FIG. 14 is a cross-sectional view of the ring cut, variable width configuration of the FIG. 13 patient end portion.

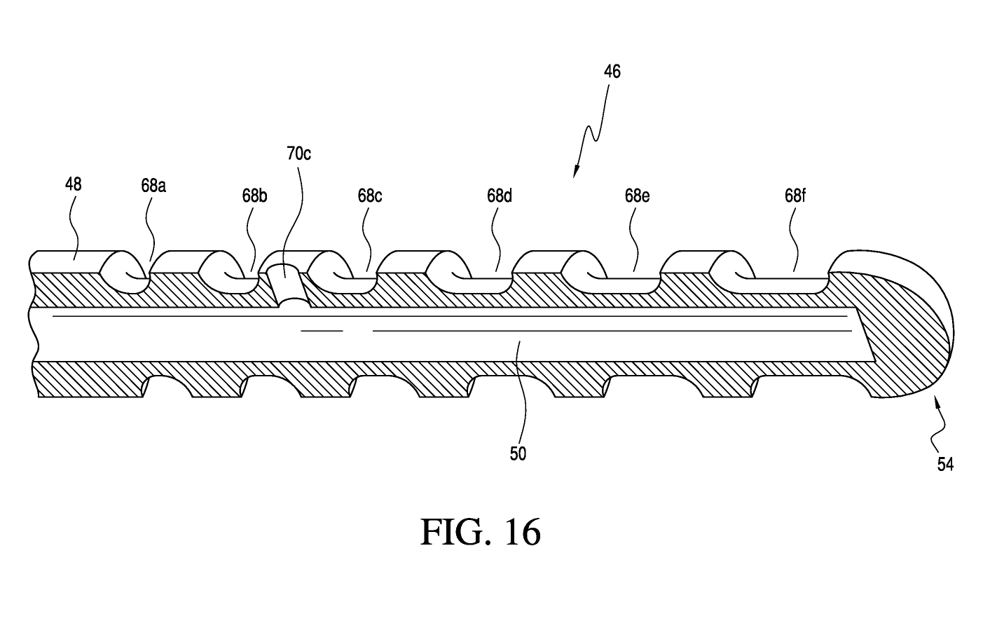

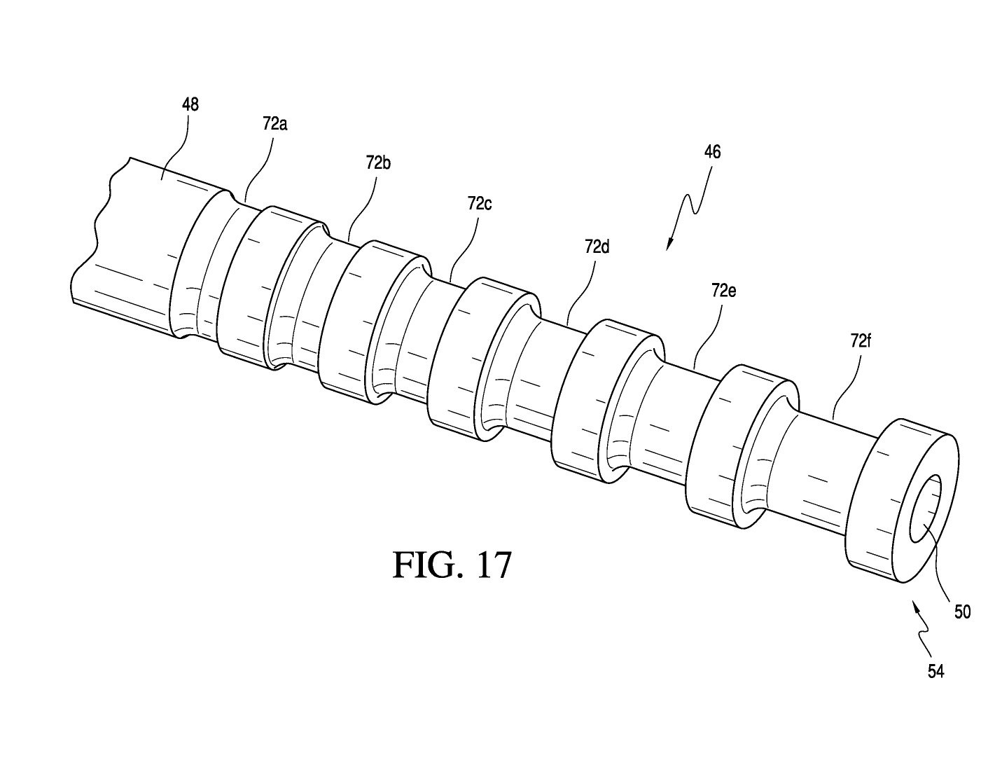

FIG. 15 shows another variant of the distal portion 46 of the inventive catheter. The FIG. 15 embodiment is also a ring cut, variable width distal portion. However, there is no opening at distal end 54. Instead, a number of side ports 70a-70c are provided for outputting the medicament. The ring cuts, with their variable width, are the same as those shown in the FIG. 14 embodiment and are therefore labeled the same. FIG. 16 is a cross-sectional view of the FIG. 15 variant of the distal portion of the inventive catheter.

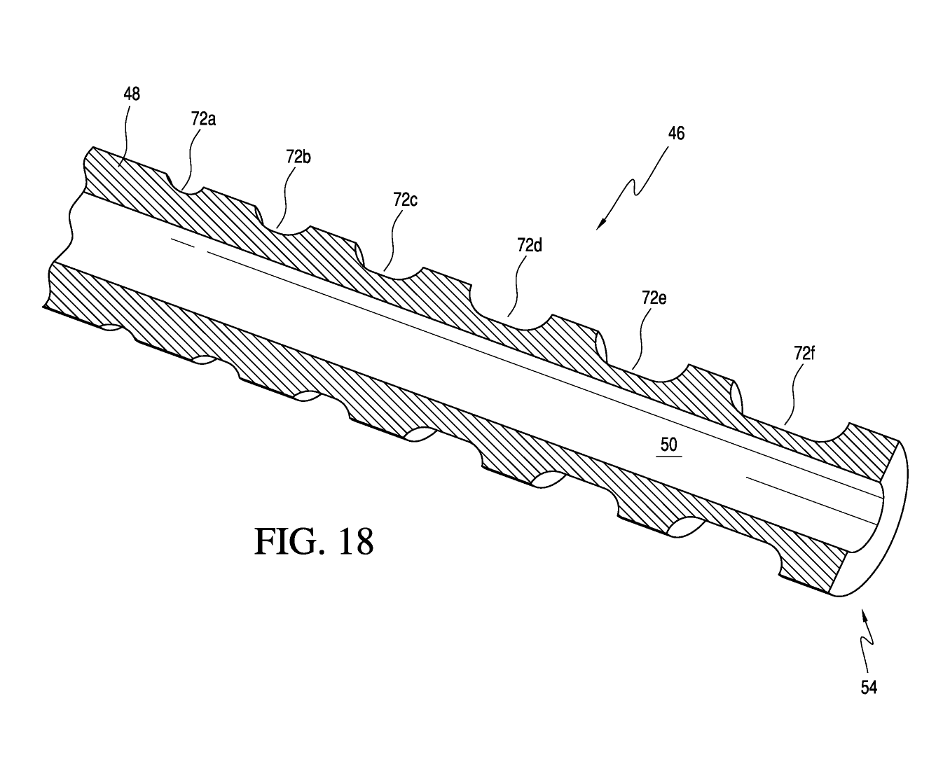

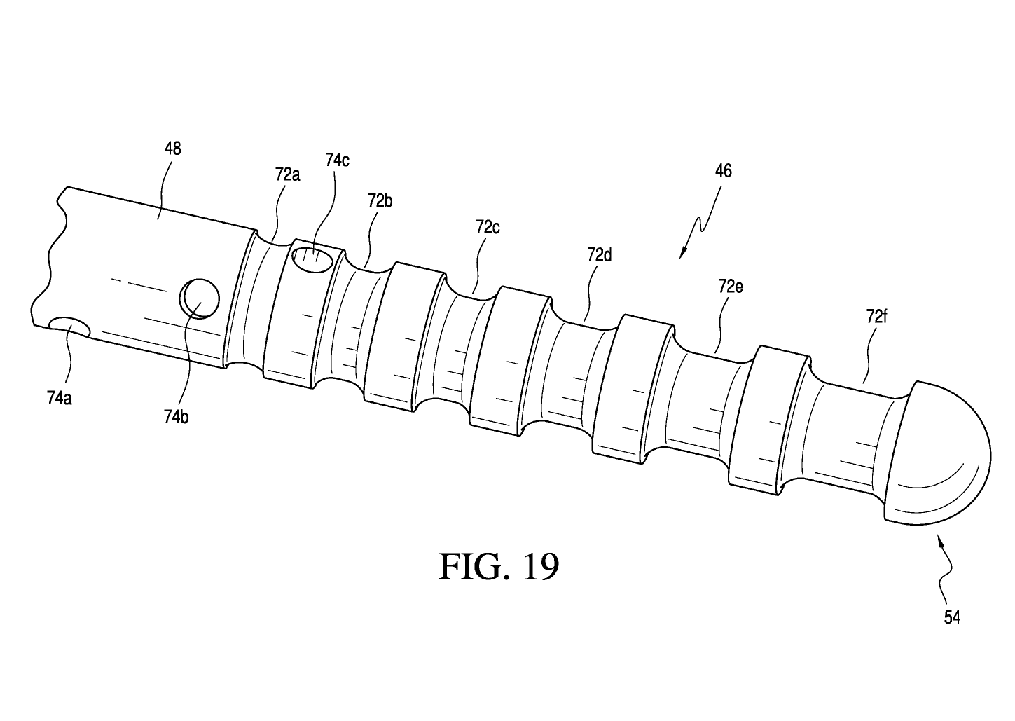

FIG. 17 is yet another variant of the patient end portion of the inventive catheter. In the FIG. 17 embodiment, distal portion 46 has a ring cut, variable width and depth configuration. With the same number of ring cuts as shown in the earlier embodiments of FIGS. 13-16, the various ring cuts 72a-72f, along with having different widths, also have different cut depths. As shown, ring cut 72a has a cut depth that is smaller than that of ring cut 72b, which in turn is smaller than ring 72c, etc. until the last ring cut 72f proximate to distal end 54, which has the deepest depth or cut into the catheter body 48. Thus configured, the FIG. 17 embodiment provides yet another variant of the patient end portion of the catheter where there is greater flexibility closer toward the distal end 54. FIG. 18 is a cross-sectional view of the FIG. 17 embodiment.

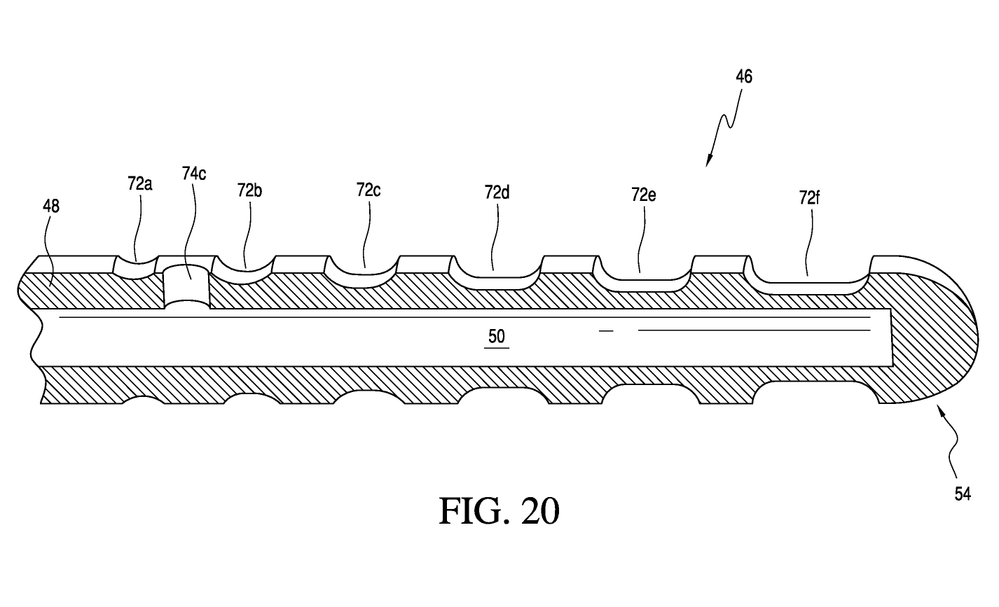

FIG. 19 is yet another variant of the distal body 46 of the inventive catheter. The FIG. 19 embodiment also has a ring cut, variable width and depth configuration like that shown FIGS. 17 and 18. But instead of having an opening at distal end 54, a number of side ports 74a-74c are provided for the FIG. 19 non-open patient end portion of the inventive catheter. FIG. 20 is a cross-sectional view of the FIG. 19 variant of the patient end portion of the inventive catheter.

It should be appreciated that the present invention is subject to many variations, modifications and changes in detail. Thus, all matters described throughout this specification and shown in the accompanying drawings should be interpreted as illustrative only and not in a limiting sense.

1-24. (canceled)

25. A catheter, comprising an elongated tube having a main body and a distal body, the tube having a circumferential wall formed from an elastomeric material defining at least one lumen terminating at a distal end having an opening to the lumen, the wall having an outer circumferential surface and an inner circumferential surface, at least one reinforcement member embedded in the wall along at least the length of the main body, the wall at the distal body having portions therealong selectively weakened to provide the distal body with flexural properties that are softer than the main body.

26. The catheter of claim 25, wherein the one reinforcement member comprises a one piece continuous strip or coil spirally embedded along at least the length of the main body.

27. The catheter of claim 25, wherein the weakened portions of the distal body result from a continuous spiral notch cut into the wall from the outer circumferential surface along the distal body.

28. The catheter of claim 25, wherein the distal body has a flexibility relative to the main body that varies in correspondence to the amount of the material removed from the weakened portions along the distal body, so that the flexibility of the distal body relative to the main body can be selectively varied along the length of the distal body.

29. The catheter of claim 25, wherein the distal body has a distal end and a proximal end that integrally extends from the main body; and
wherein the amount of material removed from the weakened portions along the length of the distal body is selectively varied between its proximal and distal ends to effect a flexibility for the distal body that increases from its proximal end to its distal end.

30. The catheter of claim 25, wherein there are a plurality of circumferential notches along the distal body, each of the notches having a different width than the other notches, the notches having respective increasing widths as the notches get closer to the distal end.

31. The catheter of claim 25, wherein there are a plurality of circumferential notches along the distal body, each of the notches having a different cut depth than the other notches, the notches having respective deeper cut depths as the notches get closer to the distal end.

32. The catheter of claim 25, wherein there are a plurality of circumferential notches along the distal body, each of the notches having a different width and cut depth than the other notches, the notches having respective wider widths and deeper cut depths as the notches get closer to the distal end.

33. The catheter of claim 25, wherein the one reinforcement member comprises a strip or coil of nonmetallic material compatible with regional anesthesia medications and adapted to be viewable under ultrasound embedded within the wall spirally along the tube.

34. The catheter of claim 25, wherein the one reinforcement member comprises a polymeric strip or coil spirally wrap about the inner circumferential wall of the tube within the wall.

35. The catheter of claim 25, wherein the one reinforcement member comprises a polymeric strip or coil made from any of polymeric materials including nylon, PET (polyester), PU (polyurethane), PEEK (polyether ether ketone), PEKK (polyether ketone ketone), PEI (polyetherimide) and polyimide.

36. A catheter comprising: an elongated tube having a main body and a distal body along a longitudinal axis, the tube having a circumferential wall formed from an elastomeric material defining at least one lumen terminating at a distal end having an opening to the lumen, the wall having an outer circumferential surface and an inner circumferential surface, at least one reinforcement member embedded in the wall of the tube, the wall at the distal body having portions of the wall therealong orthogonal to the longitudinal axis of the tube selectively weakened to thereby effect the distal body to have flexural properties that are softer than the main body.

37. The catheter of claim 36, wherein the one reinforcement member comprises a one piece continuous strip spirally embedded substantially along the length of the tube.

38. The catheter of claim 36, wherein the weakened portions at the distal body result from a continuous spiral notch cut into the wall from the outer circumferential surface along the distal body.

39. The catheter of claim 36, wherein the distal body has a flexibility relative to the main body that varies in correspondence to the amount of material removed from the weakened portions along the distal body, so that the flexibility of the distal body relative to the main body can be selectively varied along the length of the distal body.

40. The catheter of claim 36, wherein distal body has a distal end and a proximal end that integrally extends from the main body; and
wherein the amount of material removed from the weakened portions along the length of the distal body increases from the proximal end to the distal end so that the flexibility of the distal body is greater at the distal end than the proximal end.

41. The catheter of claim 36, wherein the one reinforcement member comprises a strip or coil of nonmetallic material compatible with regional anesthesia medications and adapted to be viewable under ultrasound.

42. The catheter of claim 36, wherein the one reinforcement member comprises a polymeric strip or coil spirally wrap about the inner circumferential wall of the tube within the wall.

43. The catheter of claim 36, wherein the one reinforcement member is made from any of polymeric materials including nylon, PET (polyester), PU (polyurethane), PEEK (polyether ether ketone), PEKK (polyether ketone ketone), PEI (polyetherimide) and polyimide.

44. The catheter of claim 36, wherein the one reinforcement member is not embedded in the distal body; and
wherein there are a plurality of circumferential notches along the distal body, each of the notches having a different width than the other notches, the notches having respective widths that increase toward the distal end of the distal body.

45. The catheter of claim 36, wherein the one reinforcement member is not embedded in the distal body; and
wherein there are a plurality of circumferential notches along the distal body, each of the notches having a different cut depth than the other notches, the notches having respective cut depths that increases toward the distal end of the distal body.

46. The catheter of claim 36, wherein the one reenforcement member is not embedded in the distal body; and
wherein there are a plurality of circumferential notches along the distal body, each of the notches having a different width and cut depth than the other notches, the respective widths and cut depths for the each notch being greater as the each notch gets closer toward the distal end of the distal body.

47. A method of making a catheter, comprising the steps of:
a) forming an elongated tube having a main body, a distal body and a circumferential wall formed of an elastomeric material having an outer circumferential surface and an inner circumferential surface defining at least one lumen terminating at a distal end having an opening to the lumen;
b) embedding at least one reinforcement member in the wall about at least the length of the main body; and
c) selectively weakening portions of the wall at the distal body to effect the distal body to have flexural properties that are softer than the main body;
wherein the distal body flexes relative to the main body to divert the catheter to a different direction when the distal body comes into contact with an obstacle as the catheter is maneuvered within the patient.

48. The method of claim 47, wherein the step c comprises the step of effecting a continuous spiral notch cut from the outer circumferential surface into the wall at the distal body to provide the weakened portions.

49. The method of claim 47, wherein step c comprises the step of:
varying the amount of the material removed from the weakened portions along the distal body to selectively adjust the flexibility along the distal body relative to the main body.

50. The method of claim 47, further comprising the step of:
forming the one reinforcement member as a strip or coil of nonmetallic material compatible with regional anesthesia medications and adapted to be viewable under ultrasound.

51. The method of claim 47, further comprising the step of:
forming the one reinforcement member from any of polymeric materials including EPTFE (expanded polytetrafluoroethylene), nylon, PU (polyurethane), polyurethane PET, PEEK (polyether ether ketone), PEKK (polyether ketone ketone), PEI (polyetherimide) and polyimide.

52. The method of claim 47, wherein the step c further comprises the steps of:
forming a plurality of circumferential notches along the distal body; and
forming each of the notches to have a different width than the other notches; and
increasing the respective widths of the notches as the notches get closer to the distal end.

53. The method of claim 47, wherein the step c further comprises the steps of:
forming a plurality of circumferential notches along the distal body;
forming each of the notches to have a different cut depth than the other notches;
increasing the depth of the respective notches as the notches get closer to the distal end.

54. The method of claim 47, wherein the step c comprises the steps of:
forming a plurality of circumferential notches along the distal body;
forming each of the notches to have a different width and cut depth than the other notches; and
increasing the respective widths and cut depths for the notches as the notches get closer to the distal end.
